(19) United States
(12) Patent Application Publication
(10) Pub. No.: US 2025/0043248 A1
(43) Pub. Date: Feb. 6, 2025

(54) PREPARATION METHOD AND USE OF HYDROGEL MATERIAL FOR GROWTH OF BLOOD VESSEL ORGANOIDS

(71) Applicant: Union Hospital Tongji Medical College HUST, Wuhan City (CN)

(72) Inventors: Di SUN, Wuhan City (CN); Jiaming SUN, Wuhan City (CN); Zhenxing WANG, Wuhan City (CN); Shize LEI, Wuhan City (CN)

(73) Assignee: Union Hospital Tongji Medical College HUST, Wuhan City (CN)

(21) Appl. No.: 18/409,015

(22) Filed: Jan. 10, 2024

(30) Foreign Application Priority Data
Aug. 4, 2023 (CN) 202310978630.5

(51) Int. Cl. C12N 5/071

(52) U.S. Cl. CPC C12N 5/0691; C12N 2506/45; C12N 2513/00; C12N 2533/30; C12N 2533/54

(57) ABSTRACT

The present disclosure provides a preparation method and use of a hydrogel material for growth of blood vessel organoids (BVOs). The preparation method of the hydrogel material includes: (1) synthesis of gelatin methacryloyl (GelMA), and (2) synthesis of ns-GelMA-PEO. The hydrogel material may be used for in vitro cultivation of BVOs. The hydrogel material ns-GelMA-PEO prepared by the present disclosure may significantly improve the survival and budding abilities of BVOs. The ns-GelMA-PEO provides a novel solution for in vitro cultivation of BVOs, and may well support the growth and differentiation of organoids.

### CROSS REFERENCE TO RELATED APPLICATION

This patent application claims the benefit and priority of Chinese Patent Application No. 202310978630.5 filed with the China National Intellectual Property Administration on Aug. 4, 2023, the disclosure of which is incorporated by reference herein in its entirety as part of the present application.

### TECHNICAL FIELD

The present disclosure relates to the field of biomedical tissue engineering, and specifically relates to a preparation method and use of a hydrogel material for growth of blood vessel organoids.

### BACKGROUND

Organoids are a class of microscopic three-dimensional (3D) structures that may self-assemble and are produced by stem cells cultivated in vitro, including pluripotent stem cells (PSCs) and adult stem cells (ASCs). Organoids have a size of about hundreds of micrometers to several millimeters. Organoids may replicate complicated spatial configurations similar to corresponding organs or tissues, and the spatial configurations may exhibit cell-to-cell and cell-to-matrix interactions and have similar physiological functions to differentiated tissues in vivo. BVOs refer to pseudo-natural blood vessel-like tissues with lumina and complicated branches that are produced through 3D cultivation of induced pluripotent stem cells (iPSCs) in vitro. A cell composition of BVO includes endothelial cells (ECs) and pericytes. BVOs have similar functions for synthesis of von Willebrand factor (vWF) and uptake of low-density lipoprotein (LDL) to natural tissues, and may synthesize type IV collagen, a major component of a reticular structure of a base membrane.

Currently, the in vitro cultivation of BVOs relies on Matrigel and type I collagen. Matrigel is a natural hydrogel derived from a mouse tumor tissue, and has excellent biocompatibility. However, there are the following disadvantages: (1) Inter-batch compositions of Matrigel are difficult to control, and corresponding inter-batch differences lead to inconsistent performance of stem cells. (2) Matrigel may carry pathogens or immunogens, and thus it is difficult to allow clinical transformation of cells and organoids cultivated with Matrigel. (3) Matrigel and type I collagen are very expensive, and 5 mL of Matrigel or 100 mg of type I collagen requires about 5,000 RMB. (4) During an actual operation, because Matrigel and type I collagen both are temperature-sensitive hydrogels, 2 h of crosslinking in a 37° C. incubator is required for gelatinization, which results in a high time cost and makes it impossible to allow forming by a strategy such as 3D printing. Therefore, there is an urgent need to develop a novel intelligent responsive hydrogel material as a 3D cultivation matrix for BVOs.

Gelatin methacryloyl (GelMA) is a gelatin-modified light-curable hydrogel, and a photocrosslinking process of GelMA may be completed within only tens of seconds. GelMA exhibits both excellent biocompatibility and excellent forming performance. Pores inside crosslinked GelMA are determined by distances among polymer chains within a hydrogel network, and mostly have a diameter of a nano-scale or less. Pores of this size allow the diffusion of small-molecule substances. However, the transport of large-molecule substances, the proliferation and migration of internal cells, and the infiltration of host cells often need to be completed through pores with a pore size of tens of microns. Traditional GelMA may maintain the survival of a variety of cells, but GelMA may hardly support the survival, differentiation, and vascular budding of BVOs, which are cell clusters with diversified cell components, complicated spatial structures, and large volumes (hundreds of microns). Therefore, in order to improve the survival of BVOs inside GelMA, it is necessary to further increase a pore size of GelMA and optimize a formula of GelMA without changing the mechanical properties and forming performance of GelMA.

Unlike the cultivation of conventional cells, organoids require long-term in vitro cultivation and induction. Traditional solvents for hydrogels are often buffers such as phosphate buffered saline (PBS), which may hardly provide nutrients for long-term growth and differentiation of organoids.

### SUMMARY

In view of the deficiencies in the prior art, the present disclosure provides a preparation method of a hydrogel material for growth of BVOs. The hydrogel material may improve a cultivation environment for organoids and promote the vascularization of organoids. In the novel hydrogel formula of the present disclosure, more organoid vascularization-promoting components are added to well simulate an in vivo microenvironment.

The present disclosure adopts the following technical solutions: A preparation method of a hydrogel material for growth of BVOs is provided, including the following steps:
(1) synthesis of GelMA
dissolving type A gelatin in PBS according to a mass/volume ratio of 1:(10-20) g/mL to obtain a gelatin solution: slowly adding methacrylic anhydride (MAA) dropwise to the gelatin solution, and stirring a resulting mixture at 40° C. to 60° C. to allow a reaction for 1 h to 3 h, where a mass ratio of the type A gelatin to the MAA is 1:(0.6-0.8); and subjecting a resulting reaction system to dialysis with deionized water at 40° C. for 3 d to 7 d to remove unreacted MAA and by-products, aseptically filtering a purified GelMA monomer solution through a microporous filter membrane, and lyophilizing a resulting filtrate to obtain the GelMA; and
(2) synthesis of Ns-GelMA-PEO
dissolving the GelMA obtained in the step (1), a photoinitiator of lithium phenyl (2,4,6-trimethylbenzoyl) phosphinate (LAP), and polyethylene oxide (PEO) in a mixed solvent, and filtering a resulting solution through a microporous filter membrane to obtain the ns-GelMA-PEO including 5% to 10% of the GelMA, 0.1% to 0.5% of the LAP, and 0.5% to 1.6% of the PEO in mass percentages,
where the mixed solvent is prepared from the following substances in volume percentages: 82% of PBS, 6% of 10×Dulbecco's Modified Eagle Medium (DMEM), 1.2% of N-2-hydroxyethylpiperazine-N-2-ethanesulfonic acid (HEPES), 0.6% of Glutamax, 9.2% of a Ham's F12 medium, and 1% of a NaOH solution.

Further, the microporous filter membrane is a 0.22 μm filter membrane.

Further, the PEO has a molecular weight of 7,000 kDa to 8,000 kDa.

The hydrogel material prepared by the present disclosure may be used for in vitro cultivation of BVOs.

Use of hydrogel material in BVOs is provided. An in vitro cultivation method of BVOs includes the following steps:
(1) allowing iPSCs to grow adherently on a 1% Matrigel-coated 6-well plate until a cell density reaches about 80%; adding a 0.5 mM EDTA-containing digestion solution, incubating the plate in a 37° C. incubator for 5 min to 8 min, and, removing the digestion solution when it is observed under an inverted microscope that 95% or more of cells begin to be separated, rounded, and brightened; gently resuspending the cells with a mTeSR™ 1 PSC complete medium including an ATP-competitive ROCK pathway inhibitor Y-27632 in a ratio of 1:4,000 to obtain a cell suspension; and inoculating the cell suspension into a Matrigel-coated 6-well plate, and cultivating the cells in a 37° C. and 5% CO₂ incubator, during which the original medium is replaced with a mTeSR™ 1 complete medium on day 2 to remove unadherent cells;
(2) removing the original iPSC medium, adding 0.5 mM EDTA at 0.6 mL/well to 1 mL/well for rinsing, and removing the EDTA; adding a marine-origin enzyme (Accutase) for cell dissociation at 0.6 mL/well to 1 mL/well, and incubating the plate in a 37° C. incubator for 3 min to 5 min; when cells begin to be rounded and brightened, gently pipetting a resulting system up and down to obtain single cells, and resuspending the single cells with a cell aggregation medium; and inoculating the single cells into an ultra-low attachment 6-well plate at a cell density of 2× 10⁵ to 5×10⁵/well, and cultivating the single cells in a 37° C. and 5% CO₂ incubator for 1 d to 3 d to obtain spherical iPSC aggregates each with a smooth surface and a diameter of 50 μm to 100 μm; and
(3) resuspending the spherical iPSC aggregates with a medium including an N2B27 medium, a glycogen synthase kinase 3β (GSK-3β) inhibitor CHIR99021 at 12 μM, and bone morphogenetic protein 4 (BMP4) at 30 ng/mL, and cultivating the spherical iPSC aggregates for 3 d to induce mesoderm differentiation; adding vascular endothelial growth factor A (VEGF-A) at 10 ng/ml and forskolin at 2 μM into medium, and further cultivating resulting iPSC aggregates in a 37° C. and 5% CO₂ incubator for 2 d; and embedding resulting iPSC aggregates in the ns-GelMA-PEO for 3D cultivation, irradiating the ns-GelMA-PEO with ultraviolet (UV) light for 30 s to allow curing, adding 1 mL of a StemPro-34 SFM complete medium fully pre-warmed at 37° C. to each well, and cultivating the iPSC aggregates in a 37° C. and 5% CO₂ incubator for 1 d to 3 d to allow vascular budding to form a vascular network.

Further, in the step (2), the cell aggregation medium is prepared from the following substances in volume percentages: 77% of a KnockOut DMEM/F12 medium, 19% of a KnockOut serum replacement, 1% of a dipeptide additive of L-alanyl-L-glutamine (Glutamax), 1% of a non-essential amino acid (NEAA) additive, 1% of a 2-mercaptoethanol (also known as β-mercaptoethanol) dilution with a dilution ratio of 1:100, and 1% of penicillin-streptomycin.

Further, in the step (3), the StemPro-34 SFM complete medium includes fetal bovine serum (FBS) in a volume percentage of 15%, the VEGF-A at 100 ng/mL, and fibroblast growth factor 2 (FGF-2) at 100 ng/mL.

The photosensitive hydrogel ns-GelMA-PEO (gelatin methacryloyl-polyethylene oxide) prepared by the present disclosure may support the growth, differentiation, and budding of BVOs at a concentration of 5%. Compared with the traditional matrix (Matrigel, type I collagen) for cultivation of BVOs, the hydrogel material of the present disclosure has a low cost and a simple composition, may undergo rapid crosslinking under UV light, and exhibits an excellent forming ability. Compared with the traditional GelMA, a pore-forming agent is added in the present disclosure to significantly expand pores inside GelMA, and thus the ns-GelMA-PEO has a large pore size, which may significantly improve the survival and budding abilities of BVOs. The ns-GelMA-PEO provides a novel solution for in vitro cultivation of BVOs.

Compared with a solvent (a buffer such as PBS) in traditional hydrogel, the present disclosure optimizes a solvent formula for a hydrogel to support a growth viability of organoids. In order to improve a cultivation environment for organoids and promote the vascularization of organoids, increased organoid vascularization-promoting components are added in the novel hydrogel formula to well simulate an in vivo microenvironment, and a solvent in the novel hydrogel formula includes 6% of 10×DMEM, 9.2% of a Ham's F12 medium, 0.6% of Glutamax, 1.2% of HEPES, and 1% of NaOH. DMEM is a widely-used basal medium, and may be used to support the growth of many types of mammalian cells. In the present disclosure, the 10×DMEM is adopted as a basic component of the solvent for the hydrogel to maintain an osmotic pressure in the hydrogel consistent with an osmotic pressure in cells while providing basic nutrition for the growth of organoids. The Ham's F12 medium provides nutrients such as zinc, putrescine, hypoxanthine, and thymidine for organoids. The Glutamax is a dipeptide (L-alanyl-L-glutamine). The Glutamax may be hydrolyzed by aminopeptidase gradually released by cells to slowly release L-alanine and L-glutamine into the hydrogel, and then the L-alanine and L-glutamine may be absorbed by cells for protein production and tricarboxylic acid (TCA) cycle. Therefore, the Glutamax may provide essential amino acids (EAAs) for cells during the long-term in vitro cultivation and differentiation of organoids, thereby improving a viability and a lifespan of the organoids. The HEPES (N-2-Hydroxyethylpiperazine-N-2-ethanesulfonic acid) is a zwitterionic organic chemical buffer. Because the cultivation and induction of organoids involves complicated operations and requires a long-term treatment of cells outside a CO₂ incubator, the HEPES needs to be added to provide an additional buffering capacity for the hydrogel. The NaOH is added at a small amount to adjust a pH to keep the hydrogel neutral. Compared with a hydrogel PBS-GelMA-PEO prepared with the conventional solvent PBS, the hydrogel ns-GelMA-PEO prepared with the solvent of the present disclosure may well support the growth and differentiation of organoids.

### BRIEF DESCRIPTION OF THE DRAWINGS

FIG. 1 is a comparison diagram of photosensitivities of the GelMA-PEO prepared in the present disclosure and GelMA;

FIG. 2 is a comparison diagram of porosities of the GelMA-PEO prepared in the present disclosure and GelMA;

FIG. 3 shows the comparison of growth statuses of organoids in different hydrogels (from left to right: Matrigel+Col I→GelMA→ns-GelMA-PEO);

FIG. 4 shows the comparison of growth statuses of organoids in GelMA-PEO hydrogels with different solvents; and

FIGS. 5A-C show a development process of BVOs in ns-GelMA-PEO (FIG. 5A: low-magnification light microscopy images on day 0, day 1, and day 5; FIG. 5B: a high-magnification light microscopy image on day 5; and FIG. 5C: an immunofluorescence staining image, where CD31-positive ECs are arranged into a reticular structure).

### DETAILED DESCRIPTION OF THE EMBODIMENTS

The present disclosure is illustrated in detail below in combination with examples to enable the advantages and features of the present disclosure to be readily understood by those skilled in the art, thereby defining the protection scope of the present disclosure definitely. Apparently, the described examples are some rather than all of the examples of the present disclosure. All other examples obtained by a person of ordinary skill in the art based on the examples of the present disclosure without creative efforts should fall within the protection scope of the present disclosure.

### Example 1

A preparation method of a hydrogel material for growth of BVOs was provided, including the following steps:
(1) 10 g of type A gelatin was dissolved in 100 mL of PBS to obtain a gelatin solution, MAA was slowly added dropwise to the gelatin solution (a mass ratio of the type A gelatin to the MAA was 1:0.6), and a resulting mixture was stirred at 50° C. to allow a reaction for 3 h; and a resulting reaction system was subjected to dialysis with deionized water at 40° C. for 5 d to remove unreacted MAA and by-products, a purified GelMA monomer solution was aseptically filtered through a 0.22 μm filter membrane and then lyophilized to obtain GelMA, and the GelMA was stored at 4° C.
(2) The GelMA obtained in the step (1), LAP (a photoinitiator), and PEO were dissolved in a mixed solvent, and a resulting solution was filtered through a 0.22 μm microporous filter membrane to obtain ns-GelMA-PEO including 5% of the GelMA, 0.2% of the LAP, and 1.6% of the PEO in mass percentages,
where the mixed solvent was prepared from the following substances in volume percentages: 82% of PBS, 6% of 10×DMEM, 1.2% of HEPES, 0.6% of Glutamax, 9.2% of a Ham's F12 medium, and 1% of a NaOH solution.

### Example 2

A preparation method of a hydrogel material for growth of BVOs was provided, including the following steps:
(1) 5 g of type A gelatin was dissolved in 100 mL of PBS to obtain a gelatin solution, MAA was slowly added dropwise to the gelatin solution (a mass ratio of the type A gelatin to the MAA was 1:0.6), and a resulting mixture was stirred at 55° C. to allow a reaction for 1 h; and a resulting reaction system was subjected to dialysis with deionized water at 40° C. for 3 d to remove unreacted MAA and by-products, a purified GelMA monomer solution was aseptically filtered through a 0.22 μm filter membrane and then lyophilized to obtain GelMA, and the GelMA was stored at 4° C.
(2) The GelMA obtained in the step (1), LAP (a photoinitiator), and PEO were dissolved in a mixed solvent, and a resulting solution was filtered through a 0.22 μm microporous filter membrane to obtain ns-GelMA-PEO including 7% of the GelMA, 0.15% of the LAP, and 1% of the PEO in mass percentages,
where the mixed solvent was prepared from the following substances in volume percentages: 82% of PBS, 6% of 10×DMEM, 1.2% of HEPES, 0.6% of Glutamax, 9.2% of a Ham's F12 medium, and 1% of a NaOH solution.

### In Vitro Cultivation of BVOs

(1) iPSCs were allowed to grow adherently on a 1% Matrigel-coated 6-well plate until a cell density reached about 80%, and then cell passage (1:2) was conducted as follows: a 0.5 mM EDTA-containing digestion solution was added, the plate was incubated in a 37° C. incubator for 5 min to 8 min, and when it was observed under an inverted microscope that 95% or more of cells began to be separated, rounded, and brightened, the digestion solution was removed; the cells were gently resuspended with a mTeSR™ 1 complete medium including Y-27632 according to a ratio of 1:4,000 to obtain a cell suspension; and the cell suspension was inoculated into a Matrigel-coated 6-well plate, and the cells were cultivated in a 37° C. and 5% CO₂ incubator, during which the original medium was replaced with a mTeSR™ 1 complete medium on day 2 to remove unadherent cells.

(2) Preparation of iPSC aggregates: The original iPSC medium was removed, and 0.5 mM EDTA was added at 0.6 mL/well for rinsing and then removed; Accutase was added at 0.6 mL/well, and the plate was incubated in a 37° C. incubator for 3 min to 5 min; when cells began to be rounded and brightened, a resulting system was gently pipetted up and down to obtain single cells; and the single cells were resuspended with an aggregation medium (including: 77% KnockOut DMEM/F12+19% KnockOut Serum Replacement+1% Glutamax+1% NEAAs+1% β-mercaptoethanol dilution with a dilution ratio of 1:100+1% penicillin-streptomycin), then inoculated into an ultra-low attachment 6-well plate at a cell density of 2×10⁵/well, and cultivated in a 37° C. and 5% CO₂ incubator for 1 d to 3 d to obtain spherical iPSC aggregates each with a smooth surface and a diameter of 50 μm to 100 μm.

(3) The spherical iPSC aggregates were resuspended with a N2B27+12 μM CHIR99021+30 ng/mL BMP-4 medium, and cultivated for 3 d to induce mesoderm differentiation; VEGF-A was added at 10 ng/mL, and forskolin was added at 2 μM; resulting iPSC aggregates were further cultivated in a 37° C. and 5% CO₂ incubator for 2 d and then embedded in a Matrigel-Collagen I mixed matrix or the ns-GelMA-PEO for 3D cultivation; the Matrigel-Collagen I was allowed to stand in an incubator for 2 h to allow curing, or the ns-GelMA-PEO was irradiated with UV light for 30 s to allow curing; and 1 mL of a StemPro-34 SFM complete medium (including: 15% FBS+100 ng/mL VEGF-A+100 ng/ml FGF-2) fully pre-warmed at 37° C. was added to each well, and the iPSC aggregates were cultivated in a 37° C. and 5% CO₂ incubator for 1 d to 3 d to allow vascular budding to form a vascular network. The growth and differentiation statuses of organoids in the two matrices were compared through light microscopy analysis and immunofluorescence staining. Matrigel-collagen I, a matrix reported in the literature for successful cultivation of BVOs, was adopted here as a positive control. The media and reagents used in the in vitro cultivation of BVOs were commercially-available products.

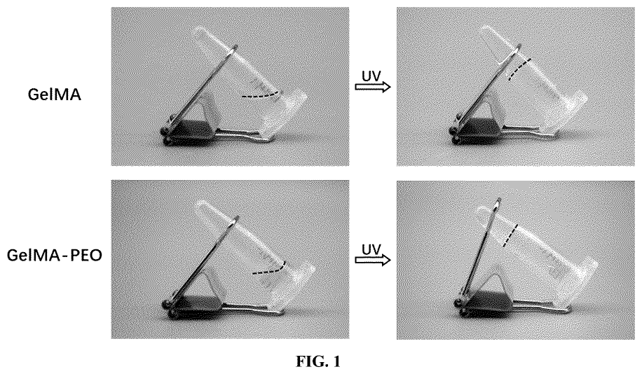

It can be seen from FIG. 1 that the GelMA-PEO prepared by the present disclosure has excellent photosensitivity, and may undergo crosslinking under UV light.

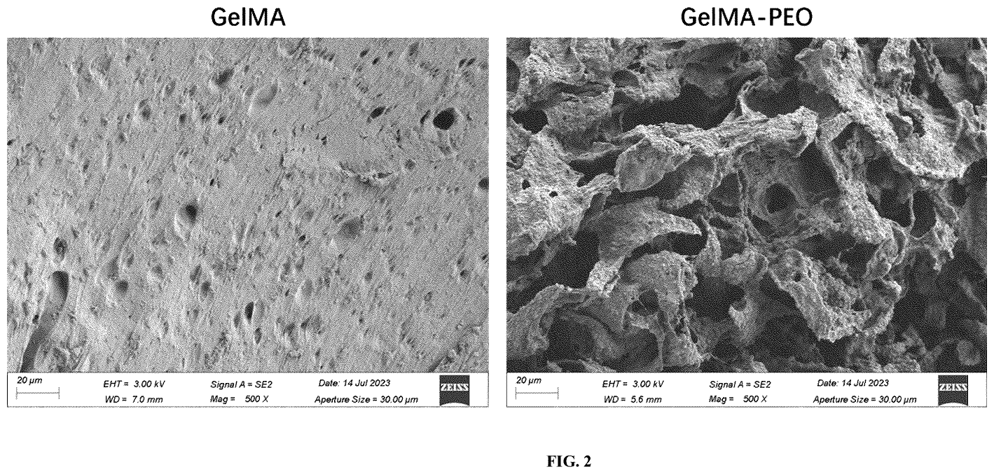

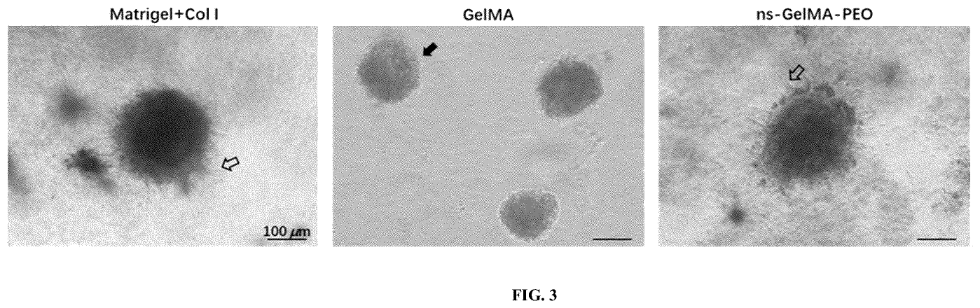

It can be seen from FIG. 2 that the GelMA has a dense structure and a low porosity; and the GelMA-PEO has a loose structure, a high porosity, and a large number of interconnected pores.

It can be seen from FIG. 3 that BVOs in the ns-GelMA-PEO and BVOs in the Matrigel+Col I both undergo excellent proliferation, and have a size of about 200 μm and an obvious vascular budding effect at an edge (shown by a hollow arrow); and cell spheroids in the GelMA have a smooth edge, no obvious vascular budding, and a small size (shown by a solid arrow).

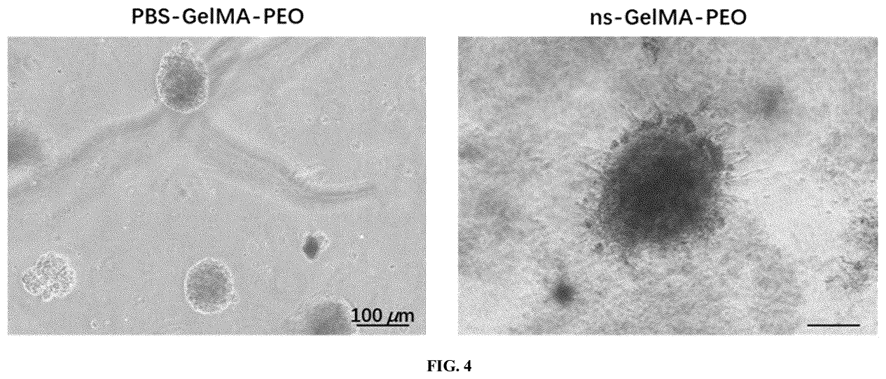

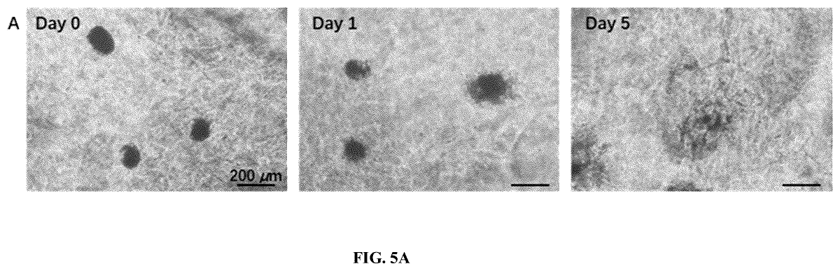

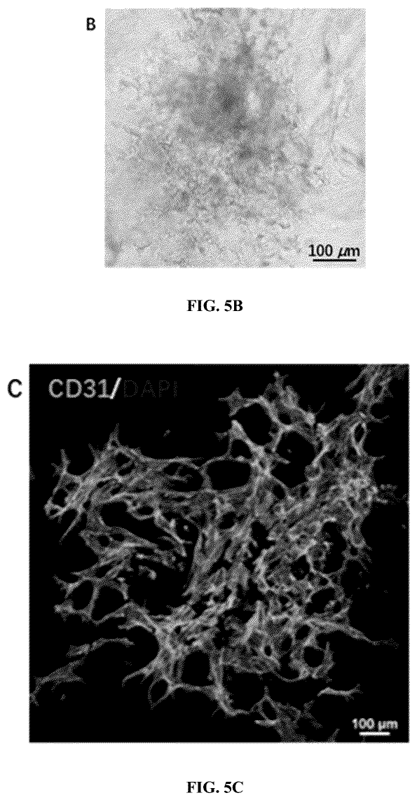

It can be seen from FIG. 4 that BVOs in the hydrogel GelMA-PEO prepared with the solvent of the present disclosure (ns-GelMA-PEO) have a large size and an obvious vascular budding effect at an edge; and cell spheroids in the hydrogel GelMA-PEO prepared with PBS (PBS-GelMA-PEO) have a small size and no obvious vascular budding.

Compared with the hydrogel PBS-GelMA-PEO prepared with the conventional solvent PBS, the hydrogel ns-GelMA-PEO prepared with the solvent of the present disclosure may well support the growth and differentiation of organoids.

The above are merely specific implementations of the present disclosure, and the protection scope of the present disclosure is not limited thereto. Any modification or replacement easily conceived by those skilled in the art within the technical scope of the present disclosure should fall within the protection scope of the present disclosure. Therefore, the protection scope of the present disclosure should be subject to the protection scope of the claims.

What is claimed is:

1. A method for preparing a hydrogel material for growth of blood vessel organoids (BVOs), comprising the following steps:
(1) synthesis of gelatin methacryloyl (GelMA)
dissolving type A gelatin in phosphate buffered saline (PBS) according to a mass/volume ratio of 1:(10-20) g/mL to obtain a gelatin solution: slowly adding methacrylic anhydride (MAA) dropwise to the gelatin solution, and stirring a resulting mixture at 40° C. to 60° C. to allow a reaction for 1 h to 3 h, wherein a mass ratio of the type A gelatin to the MAA is 1:(0.6-0.8); and subjecting a resulting reaction system to dialysis with deionized water at 40° C. for 3 d to 7 d to remove unreacted MAA and by-products, aseptically filtering a purified GelMA monomer solution through a microporous filter membrane, and lyophilizing a resulting filtrate to obtain the GelMA; and
(2) synthesis of ns-GelMA-PEO
dissolving the GelMA obtained in the step (1), a photoinitiator of lithium phenyl (2,4,6-trimethylbenzoyl) phosphinate (LAP), and polyethylene oxide (PEO) in a mixed solvent, and filtering a resulting solution through a microporous filter membrane to obtain the ns-GelMA-PEO comprising 5% to 10% of the GelMA, 0.1% to 0.5% of the LAP, and 0.5% to 1.6% of the PEO in mass percentages,
wherein the mixed solvent is prepared from the following substances in volume percentages: 82% of PBS, 6% of 10×Dulbecco's Modified Eagle Medium (DMEM), 1.2% of N-2-hydroxyethylpiperazine-N-2-ethanesulfonic acid (HEPES), 0.6% of Glutamax, 9.2% of a Ham's F12 medium, and 1% of a NaOH solution.

2. The method according to claim 1, wherein the microporous filter membrane is a 0.22 μm filter membrane.

3. The method according to claim 1, wherein the PEO has a molecular weight of 7,000 kDa to 8,000 kDa.

4. A method for cultivating BVOs, wherein comprising cultivating BVOs by using hydrogel material according to claim 1 in vitro.

5. The method according to claim 4, comprising the following steps:
(1) allowing induced pluripotent stem cells (iPSCs) to grow adherently on a 1% Matrigel-coated 6-well plate until a cell density reaches about 80%; adding a 0.5 mM EDTA-containing digestion solution, incubating the plate in a 37° C. incubator for 5 min to 8 min, and when it is observed under an inverted microscope that 95% or more of cells begin to be separated, rounded, and brightened, removing the digestion solution; gently resuspending the cells with a mTeSR™ 1 pluripotent stem cell (PSC) complete medium comprising an ATP-competitive ROCK pathway inhibitor Y-27632 according to a ratio of 1:4,000 to obtain a cell suspension; and inoculating the cell suspension into a Matrigel-coated 6-well plate, and cultivating the cells in a 37° C. and 5% CO₂ incubator, during which the original medium is replaced with a mTeSR™ 1 complete medium on day 2 to remove unadherent cells;
(2) removing the original iPSC medium, adding 0.5 mM EDTA at 0.6 mL/well to 1 mL/well for rinsing, and removing the EDTA; adding a marine-origin enzyme for cell dissociation at 0.6 mL/well to 1 mL/well, and incubating the plate in a 37° C. incubator for 3 min to 5 min; when cells begin to be rounded and brightened, gently pipetting a resulting system up and down to obtain single cells, and resuspending the single cells with a cell aggregation medium; and inoculating the single cells into an ultra-low attachment 6-well plate at a cell density of 2× 10⁵ to 5×10⁵/well, and cultivating the single cells in a 37° C. and 5% CO₂ incubator for 1 d to 3 d to obtain spherical iPSC aggregates each with a smooth surface and a diameter of 50 μm to 100 μm; and
(3) resuspending the spherical iPSC aggregates with a medium comprising an N2B27 medium, a glycogen synthase kinase 3β (GSK-3β) inhibitor CHIR99021 at 12 μM, and bone morphogenetic protein 4 (BMP4) at 30 ng/mL, and cultivating the spherical iPSC aggregates for 3 d to induce mesoderm differentiation; adding vascular endothelial growth factor A (VEGF-A) at 10 ng/ml and forskolin at 2 μM, and further cultivating resulting iPSC aggregates in a 37° C. and 5% CO₂ incubator for 2 d; and embedding resulting iPSC aggregates in the ns-GelMA-PEO for three-dimensional (3D) cultivation, irradiating the ns-GelMA-PEO with ultraviolet (UV) light for 30 s to allow curing, adding 1 mL of a StemPro-34 SFM complete medium fully pre-warmed at 37° C. to each well, and cultivating the iPSC aggregates in a 37° C. and 5% CO₂ incubator for 1 d to 3 d to allow vascular budding to form a vascular network.

6. The method according to claim 5, wherein in the step (2), the cell aggregation medium is prepared from the following substances in volume percentages: 77% of a KnockOut DMEM/F12 medium, 19% of a KnockOut serum replacement, 1% of a dipeptide additive of L-alanyl-L-glutamine, 1% of a non-essential amino acid (NEAA) additive, 1% of a 2-mercaptoethanol dilution with a dilution ratio of 1:100, and 1% of penicillin-streptomycin.

7. The method according to claim 5, wherein in the step (3), the StemPro-34 SFM complete medium comprises fetal bovine serum (FBS) in a volume percentage of 15%, the VEGF-A at 100 ng/mL, and fibroblast growth factor 2 (FGF-2) at 100 ng/mL.
